(19) United States
(12) Patent Application Publication
(10) Pub. No.: US 2015/0105769 A1
(43) Pub. Date: Apr. 16, 2015

(54) Method for endoscopic treatment

(71) Applicant: OLYMPUS MEDICAL SYSTEMS CORP., Tokyo (JP)

(72) Inventors: Makoto IGARASHI, Tokyo (JP); Satoshi TAKEKOSHI, Tokyo (JP)

(73) Assignee: OLYMPUS MEDICAL SYSTEMS CORP., Tokyo (JP)

(21) Appl. No.: 14/053,947

(22) Filed: Oct. 15, 2013

(51) Int. Cl. A61B 17/32; A61B 18/08; A61B 17/08; A61N 5/06

(52) U.S. Cl. CPC A61B 17/320016; A61N 5/0603; A61B 18/082; A61B 17/083; A61N 2005/0662; A61B 2018/00589
USPC 606/40; 607/88; 606/170; 606/158

(57) ABSTRACT

A method for endoscopic treatment that performs treatment on a subject under an endoscope includes irradiating the subject with white light, performing predetermined treatment on a living tissue of the subject after irradiation with the white light, and switching from irradiation with the white light to irradiation of the subject with narrow band light having a predetermined peak wavelength according to a condition of bleeding from the living tissue in the predetermined treatment.

### BACKGROUND OF THE INVENTION

1. Field of the Invention

The present invention relates to a method for endoscopic treatment.

2. Description of the Related Art

Conventionally, various minimally invasive inspections and operations using an endoscope are performed in the medical field. Operators can insert an endoscope into a body cavity, observe an object, images of which are picked up by an image pickup apparatus provided at a distal end portion of an endoscope insertion portion and perform treatment on a lesioned region as required using a treatment instrument inserted in a treatment instrument channel. Surgery using an endoscope does not require abdominal operation or the like, thus having an advantage of reducing physical burden on a patient.

An endoscope apparatus is configured by including an endoscope, an image processing apparatus connected to the endoscope and an observation monitor. An image pickup device provided at the distal end portion of the endoscope insertion portion picks up an image of the lesioned region and the image is displayed on the monitor. The operator can perform diagnosis or necessary treatment while watching the image displayed on the monitor.

Furthermore, some endoscope apparatuses are able to perform not only normal observation using white light but also special light observation using special light such as infrared light for observation of blood vessels inside.

In the case of an infrared endoscope apparatus, for example, indocyanine green (ICG) having an absorption peak characteristic in near-infrared light in the vicinity of a wavelength of 805 nm is injected as medicine into the blood of the patient. The object is then irradiated with infrared light in the vicinity of a wavelength of 805 nm and in the vicinity of 930 nm from a light source apparatus by time sharing. A signal of an object image picked up by a CCD is inputted to a processor of the infrared endoscope apparatus.

Regarding such an infrared endoscope apparatus, there is a proposal on an apparatus whose processor assigns an image in the vicinity of a wavelength of 805 nm to a green color signal (G), an image in the vicinity of a wavelength of 930 nm to a blue color signal (B), and outputs the signals to a monitor (e.g., see Japanese Patent Application Laid-Open Publication No. 2000-41942). Since the image of infrared light in the vicinity of 805 nm which is more absorbed by the ICG is assigned to the green color, the operator can observe the infrared image with good contrast when the ICG is administered.

For example, in endoscopic submucosal dissection (hereinafter, referred to as “ESD”) using an endoscope to perform incision in a mucous membrane layer where a lesioned region exists and dissect the submucosa or the like, the operator needs to check the position of a relatively thick blood vessel in the mucous membrane so as not to cut the blood vessel by an electric knife or the like, and perform treatment such as incision.

Furthermore, endoscope apparatuses using narrow band light whose center wavelength is 415 nm and 540 nm are also being put to practical use. Using an endoscope apparatus using such narrow band light allows capillary vessels in a shallow layer below the living tissue to be displayed on a monitor.

### SUMMARY OF THE INVENTION

A method for endoscopic treatment according to an aspect of the present invention is a method for endoscopic treatment that performs treatment on a subject under an endoscope, the method including irradiating the subject with white light, performing predetermined treatment on a living tissue of the subject after irradiation with the white light, and switching from irradiation with the white light to irradiation of the subject with narrow band light having a predetermined peak wavelength according to a condition of bleeding from the living tissue in the predetermined treatment.

A method for endoscopic treatment according to another aspect of the present invention is a method for endoscopic treatment that performs treatment on a subject under an endoscope, the method including irradiating the subject with white light, and switching, based on presence or absence of pulsatile bleeding after irradiation with the white light, from irradiation with the white light to irradiation of the subject with narrow band light having a peak wavelength in spectral characteristics in a red band of a visible range between a wavelength band including a maximum value and a wavelength band including a minimum value in hemoglobin light absorption characteristics of a living tissue of the subject.

A method for endoscopic treatment according to a further aspect of the present invention is a method for endoscopic treatment that performs treatment on a subject under an endoscope, the method including irradiating the subject with white light, performing predetermined treatment on a living tissue of the subject after irradiation with the white light, and switching, after the predetermined treatment, from irradiation with the white light to irradiation of the subject with narrow band light having a predetermined peak wavelength.

### BRIEF DESCRIPTION OF THE DRAWINGS

FIG. 1 is a configuration diagram illustrating a configuration of an endoscope apparatus used for a method for endoscopic treatment according to a first embodiment of the present invention;

FIG. 2 is a diagram illustrating a configuration of a rotating filter 14 according to the first embodiment of the present invention;

FIG. 3 is a diagram illustrating an overall processing flow in narrow band light observation according to the first embodiment of the present invention;

FIG. 4 is a diagram illustrating light absorption characteristics of venous blood according to the first embodiment of the present invention;

FIG. 5 is a flowchart illustrating a flow example of the method for endoscopic treatment in ESD according to the first embodiment of the present invention;

FIG. 6 is a diagram illustrating a situation in which a distal end portion of an insertion portion 3a of an endoscope 3 according to the first embodiment of the present invention is moved close to a lesioned region AA and the lesioned region AA is included within a range of field of view of the endoscope 3;

FIG. 7 is a diagram illustrating a situation in which a pigment Pg is sprayed over the surface of the lesioned region AA according to the first embodiment of the present invention;

FIG. 8 is a diagram illustrating an example of marking according to the first embodiment of the present invention;

FIG. 9 is a diagram illustrating hemostasis treatment when bleeding occurs during marking according to the first embodiment of the present invention;

FIG. 10 is a diagram illustrating treatment of mucosal incision through local injection according to the first embodiment of the present invention;

FIG. 11 is a diagram illustrating hemostasis treatment when bleeding occurs during mucosal incision according to the first embodiment of the present invention;

FIG. 12 is a diagram illustrating submucosal dissection treatment according to the first embodiment of the present invention;

FIG. 13 is a diagram illustrating post-operation hemostasis treatment according to the first embodiment of the present invention;

FIG. 14 is a flowchart illustrating a flow example of the method for endoscopic treatment in cerebral aneurysm clipping according to a second embodiment of the present invention;

FIG. 15 is a diagram illustrating a cerebral aneurysm CA developing on a blood vessel BV and a clip CL according to the second embodiment of the present invention;

FIG. 16 is a diagram illustrating a case where the clip CL has been correctly attached to a neck NP of the cerebral aneurysm CA according to the second embodiment of the present invention;

FIG. 17 is a diagram illustrating a situation in which clipping with the clip CL is only applied up to a midpoint of the neck NP of the cerebral aneurysm CA according to the second embodiment of the present invention;

FIG. 18 is a diagram illustrating a situation in which clipping with the clip CL is applied not to the neck NP of the cerebral aneurysm CA but to the blood vessel BV according to the second embodiment of the present invention;

FIG. 19 is a flowchart illustrating a flow example of the method for endoscopic treatment in polypectomy of a large intestine according to the second embodiment of the present invention;

FIG. 20 is a diagram illustrating polypectomy of the large intestine according to the second embodiment of the present invention;

FIG. 21 is a diagram illustrating a relationship between wavelength and intensity of band-limited light including narrow band light having one predetermined peak wavelength and having a broad range;

FIG. 22 is a diagram illustrating a relationship between wavelength and intensity of band-limited light including narrow band light having two predetermined peak wavelengths and having a broad range; and

FIG. 23 is a diagram illustrating a relationship between wavelength and intensity of band-limited light including narrow band light having one predetermined peak wavelength and one ray of wide band light.

### DETAILED DESCRIPTION OF THE PREFERRED EMBODIMENTS

Hereinafter, embodiments of the present invention will be described with reference to the accompanying drawings.

### First Embodiment

### 1. Configuration of Endoscope Apparatus

Hereinafter, an embodiment of the present invention will be described with reference to the accompanying drawings.

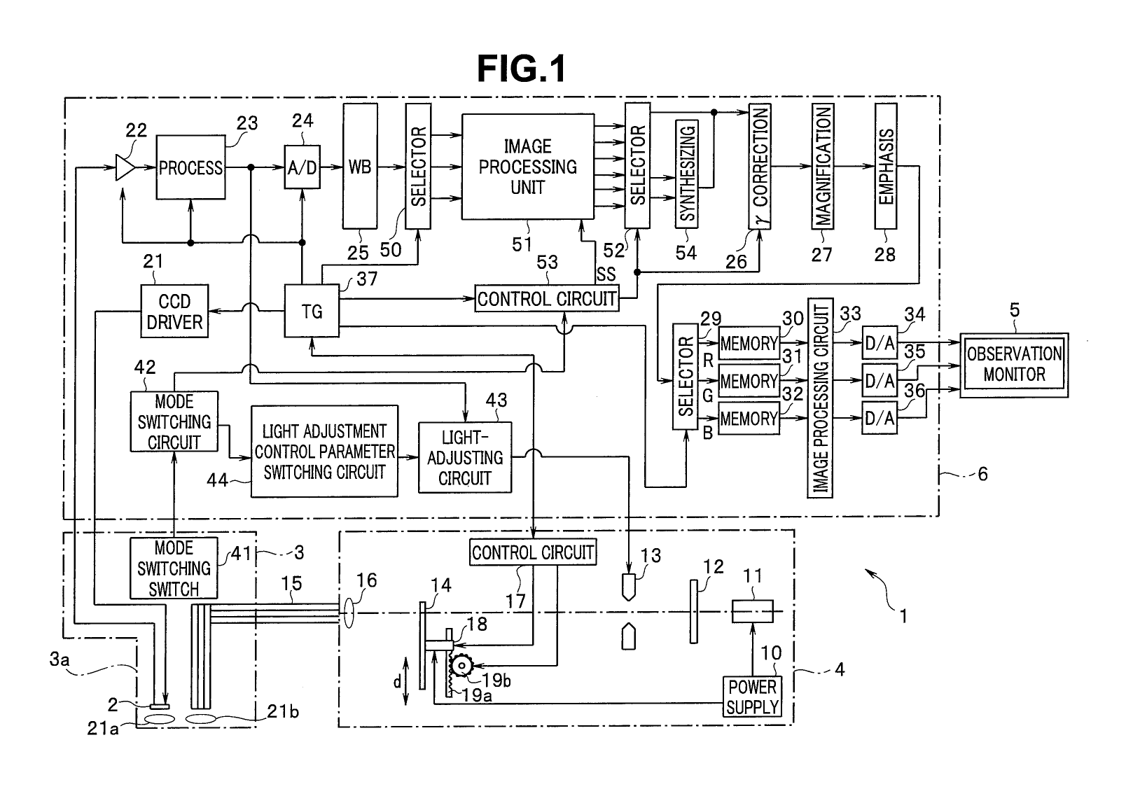

FIG. 1 is a configuration diagram illustrating a configuration of an endoscope apparatus used for a method for endoscopic treatment according to the present embodiment.

As shown in FIG. 1, an endoscope apparatus 1 of the present embodiment is constructed of an electronic endoscope 3 having a CCD 2 which is an image pickup device as a physiological image information acquiring section inserted into a body cavity to pick up an image of a tissue in the body cavity, a light source apparatus 4 that supplies illuminating light to the electronic endoscope (hereinafter, also simply referred to as “endoscope”) 3, and a video processor 6 that applies signal processing to an image pickup signal from the CCD 2 of the electronic endoscope 3 and displays an endoscopic image on an observation monitor 5. The endoscope apparatus 1 includes two modes: a normal light observation mode and a narrow band light observation mode. Note that in the following description, since the normal light observation mode of the endoscope apparatus 1 is the same as a conventional normal light observation mode, description of the configuration of the normal light observation mode is omitted and the narrow band light observation mode will be mainly described.

The endoscope 3 includes an elongated insertion portion 3a and a bending portion (not shown) is provided on a distal end side of the insertion portion 3a. The insertion portion 3a includes a distal end rigid portion on a distal end side of the bending portion and the distal end rigid portion is provided with the CCD 2. The CCD 2 constitutes an image pickup section that receives returning light of illuminating light radiated onto a subject and picks up an image of the subject. A forceps channel is provided in the insertion portion as a treatment instrument insertion channel.

The light source apparatus 4 as an illumination section is configured by including a xenon lamp 11 that emits illuminating light (white light), a heat radiation cut filter 12 that cuts heat radiation of the white light, a diaphragm apparatus 13 that controls light quantity of the white light via the heat radiation cut filter 12, a rotating filter 14 as a band-limiting section that transforms the illuminating light into frame-sequential light, a condensing lens 16 that condenses the frame-sequential light onto a plane of incidence of a light guide 15 provided in the endoscope 3 via the rotating filter 14 and a control circuit 17 that controls the rotation and position of the rotating filter 14. The xenon lamp 11, the rotating filter 14 and the light guide 15 constitute an irradiation section that irradiates the subject with illuminating light.

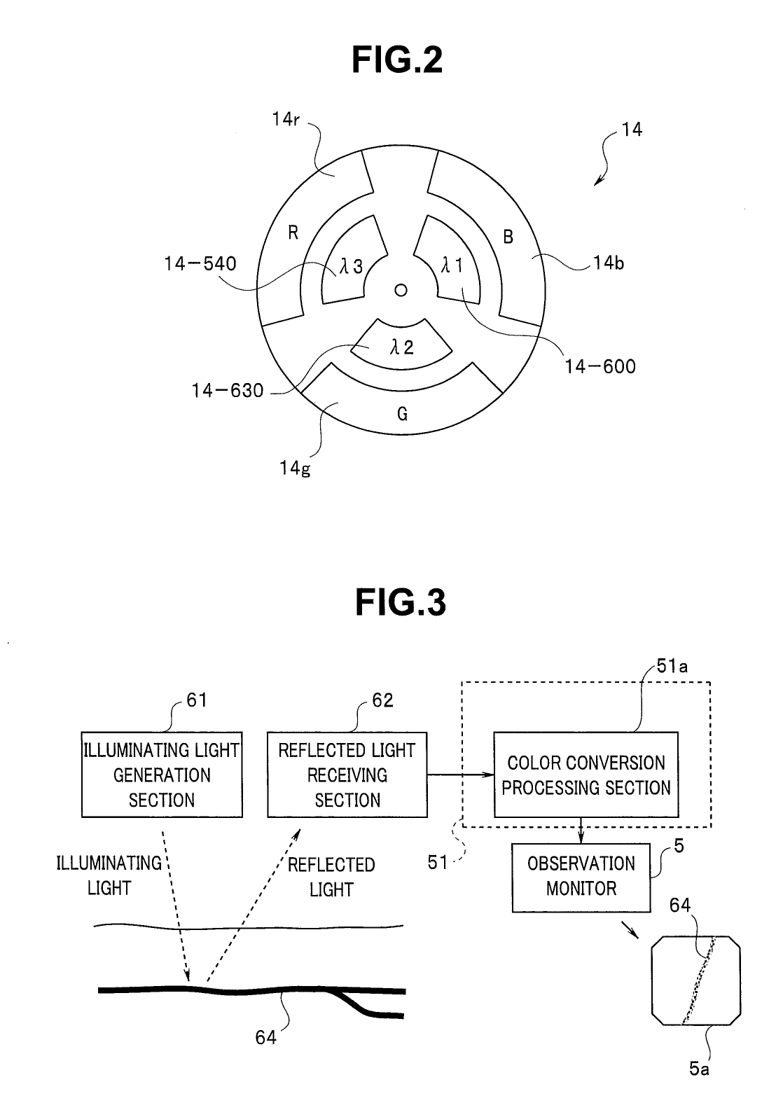

FIG. 2 is a diagram illustrating a configuration of the rotating filter 14. The rotating filter 14 is a filter that allows light from the xenon lamp 11 which is a light source to pass therethrough. The rotating filter 14 as a wavelength band-limiting section is configured into a disk shape as shown in FIG. 2, has a structure whose center is the axis of rotation and includes two filter groups. An R (red) filter section 14r, a G (green) filter section 14g and a B (blue) filter section 14b constituting a filter set to output frame-sequential light having spectral characteristics for normal light observation are arranged along a circumferential direction on an outer circumferential side of the rotating filter 14 as a first filter group.

Three filters 14-600, 14-630 and 14-540 that allow three light beams of predetermined narrow band wavelengths to pass therethrough are arranged along a circumferential direction on an inner circumferential side of the rotating filter 14 as a second filter group.

The filter 14-600 is configured so as to allow narrow band light in the vicinity of a wavelength of 600 nm (λ1) to pass therethrough as band-limited light. The filter 14-630 is configured so as to allow narrow band light in the vicinity of a wavelength of 630 nm (λ2) to pass therethrough as band-limited light. The filter 14-540 is configured so as to allow narrow band light in the vicinity of a wavelength of 540 nm (λ3) to pass therethrough as band-limited light.

Here, the term “vicinity” in the case of in the vicinity of a wavelength of 600 nm means narrow band light having a center wavelength of 600 nm and a width with a range of distribution of, for example, 20 nm centered on the wavelength of 600 nm (that is, from wavelength 590 nm to 610 nm around the wavelength of 600 nm). The same applies to the other wavelengths: wavelength 630 nm and wavelength 540 nm which will be described later.

The rotating filter 14 is arranged on an optical path from the xenon lamp 11 which is an illuminating light emitting section to an image pickup surface of the CCD 2 to place a limit on at least one (three here) of a plurality of wavelength bands of the illuminating light in each mode so as to narrow the wavelength bands.

The control circuit 17 then controls a motor 18 to rotate the rotating filter 14 and controls the rotation of the rotating filter 14.

A rack 19a is connected to the motor 18, a motor (not shown) is connected to a pinion 19b, and the rack 19a is threadably mounted on the pinion 19b. The control circuit 17 controls the rotation of the motor connected to the pinion 19b, and can thereby move the rotating filter 14 in a direction shown by an arrow d. Thus, the control circuit 17 controls the motor connected to the pinion 19b so as to place the first filter group in a normal light observation mode and the second filter group in a narrow band light observation mode on an optical path in accordance with a mode switching operation by a user, which will be described later.

Note that power is supplied to the xenon lamp 11, the diaphragm apparatus 13, the rotating filter motor 18 and the motor (not shown) connected to the pinion 19b from a power supply section 10.

Thus, the light source apparatus 4 constitutes an illumination section that irradiates the subject with at least one or more illuminating light beams (three band-limited light beams, here) having predetermined wavelength bands in the narrow band light observation mode. Here, one of the three illuminating light beams is a narrow band light beam to clearly display a blood vessel in a depth of 1 to 2 mm from a surface layer portion of a mucous membrane, and the remaining two are a narrow band light beam to display a deeper blood vessel and a narrow band light beam to display a capillary vessel in a range near the surface layer portion. For this reason, the light source apparatus 4 is an illumination apparatus that radiates at least one or more illuminating light beams via the band-limiting section that limits light to a first wavelength band (which will be described later) in the narrow band light observation mode.

The video processor 6 is configured by including a CCD drive circuit 21 which is a CCD driver, an amplifier 22, a process circuit 23, an A/D converter 24, a white balance circuit (hereinafter, referred to as “WB”) 25, a selector 50, an image processing unit 51, a selector 52, a γ correction circuit 26, a magnification circuit 27, an emphasis circuit 28, a selector 29, synchronization memories 30, 31 and 32, an image processing circuit 33, D/A converters 34,35 and 36, a timing generator (hereinafter, referred to as “TG”) 37, a mode switching circuit 42, a light-adjusting circuit 43, a light adjustment control parameter switching circuit 44, a control circuit 53, and a synthesizing circuit 54 as a display image generation section.

The CCD drive circuit 21 is intended to drive the CCD 2 provided in the endoscope 3 and output a frame-sequential image pickup signal synchronized with the rotation of the rotating filter 14 to the CCD 2. Furthermore, the amplifier 22 is intended to amplify a frame-sequential image pickup signal obtained by the CCD 2 picking up an image of a tissue in the body cavity via an objective optical system 21a provided at a distal end of the endoscope 3. Furthermore, an illumination optical system 21b is provided on a distal end side of the light guide 15.

Note that polarizing plates in a crossed Nichol state may be arranged on a front surface of the CCD 2 which is an image pickup device and on a front surface of the light guide 15 respectively. The two polarizing plates in a crossed Nichol state allow the CCD 2 to pick up an image of only light from a mucous membrane depth without receiving reflected light from the mucous membrane surface.

The process circuit 23 performs correlated double sampling and noise cancellation or the like on the frame-sequential image pickup signal via the amplifier 22. The A/D converter 24 converts the frame-sequential image pickup signal that has passed through the process circuit 23 to a digital frame-sequential image signal.

The WB 25 performs gain adjustment and white balance processing on the frame-sequential image signal digitized by the A/D converter 24 so that the brightness of an R signal of the image signal is equivalent to the brightness of a B signal of the image signal with reference to a G signal of the image signal, for example.

Note that the white balance adjustment in the WB 25 is performed with reference to the luminance of returning light of narrow band light in the vicinity of a wavelength of 600 nm.

The selector 50 assigns and outputs the frame-sequential image signal from the WB 25 into respective sections in the image processing unit 51.

The image processing unit 51 is an image signal processing section that converts an RGB image signal for normal light observation or three image signals for narrow band light observation from the selector 50 to image signals for display. The image processing unit 51 outputs image signals in a normal light observation mode and a narrow band light observation mode to the selector 52 according to a selection signal SS from the control circuit 53 based on a mode signal.

The selector 52 sequentially outputs the image signal for normal light observation and the image signal for narrow band light observation from the image processing unit 51 to the γ correction circuit 26 and the synthesizing circuit 54.

The γ correction circuit 26 applies γ correction processing to the frame-sequential image signal from the selector 52 or the synthesizing circuit 54. The magnification circuit 27 performs magnification processing on the frame-sequential image signal subjected to the γ correction processing in the γ correction circuit 26. The emphasis circuit 28 applies contour emphasis processing to the frame-sequential image signal subjected to the magnification processing in the magnification circuit 27. The selector 29 and the synchronization memories 30, 31 and 32 are intended to synchronize the frame-sequential image signals from the emphasis circuit 28.

The image processing circuit 33 reads the respective frame-sequential image signals stored in the synchronization memories 30, 31 and 32 and performs moving image color drift correction processing or the like. The D/A converters 34, 35 and 36 convert the image signals from the image processing circuit 33 to RGB analog video signals and outputs the signals to the observation monitor 5. The TG 37 receives a synchronization signal which is synchronized with the rotation of the rotating filter 14 from the control circuit 17 of the light source apparatus 4 and outputs various timing signals to the respective circuits in the video processor 6.

Furthermore, the endoscope 3 is provided with a mode switching switch 41 for switching between the normal light observation mode and the narrow band light observation mode, and the output of the mode switching switch 41 is designed to be outputted to the mode switching circuit 42 in the video processor 6. The mode switching circuit 42 of the video processor 6 is designed to output a control signal to the light adjustment control parameter switching circuit 44 and the control circuit 53. The light-adjusting circuit 43 is designed to control the diaphragm apparatus 13 of the light source apparatus 4 based on a light adjustment control parameter from the light adjustment control parameter switching circuit 44 and the image pickup signal after passing through the process circuit 23 to perform appropriate brightness control.

The respective circuits in the video processor 6 perform predetermined processing in accordance with a specified mode. Those circuits perform processing in accordance with the normal light observation mode and the narrow band light observation mode respectively, and the observation monitor 5 displays an image for normal light observation or an image for narrow band light observation. As will be described later, in the narrow band light observation mode, the observation monitor 5 displays an image based on an image signal of a relatively thick blood vessel having a diameter on the order of 1 to 2 mm at a depth of the mucous membrane on the order of 1 to 2 mm from the surface layer portion of the mucous membrane.

### 2. Overall Processing Flow of Narrow Band Light Observation

Next, an overall approximate flow of narrow band light observation according to the present embodiment will be described briefly.

FIG. 3 is a diagram illustrating an overall processing flow in narrow band light observation according to the present embodiment.

The operator inserts the insertion portion of the endoscope into the body cavity and places the distal end portion of the endoscope insertion portion in the vicinity of a lesioned region in a normal light observation mode. To observe a relatively thick blood vessel of, for example, 1 to 2 mm in diameter, in the depth running through the muscularis propria from the submucosa, the operator operates the mode switching switch 41 to switch the observation mode of the endoscope apparatus 1 to the narrow band light observation mode.

In the narrow band light observation mode, the control circuit 17 of the endoscope apparatus 1 controls the motor connected to the pinion 19b to move the position of the rotating filter 14 so as to emit light that has passed through the second filter group from the light source apparatus 4. The control circuit 53 also controls the various circuits in the video processor 6 so as to perform image processing for observation using a narrow band wavelength.

As shown in FIG. 3, in the narrow band light observation mode, illuminating light having a narrow band wavelength is emitted from an illuminating light generation section 61 from the distal end portion of the insertion portion of the endoscope 3, and after passing through the mucous membrane layer, radiated onto the blood vessel 64 running through the submucosa and the muscularis propria. Here, the illuminating light generation section 61 is configured by including the light source apparatus 4, the rotating filter 14 and the light guide 15 or the like, and emits illuminating light from the distal end of the endoscope insertion portion. As the rotating filter 14 rotates, narrow band light in the vicinity of a wavelength of 600 nm, narrow band light in the vicinity of a wavelength of 630 nm and narrow band light in the vicinity of a wavelength of 540 nm are consecutively and sequentially emitted from the light source apparatus 4 as band-limited light beams and radiated onto the object.

Reflected light beams of the narrow band light in the vicinity of a wavelength of 600 nm, the narrow band light in the vicinity of a wavelength of 630 nm and the narrow band light in the vicinity of a wavelength of 540 nm are respectively received by a reflected light receiving section 62 which is the CCD 2. The CCD 2 outputs image pickup signals of the respective reflected light beams and supplies the image pickup signals to the selector 50 via the amplifier 22 or the like. The selector 50 maintains a first image signal P1 in the vicinity of a wavelength of 600 nm, a second image signal P2 in the vicinity of a wavelength of 630 nm and a third image signal P3 in the vicinity of a wavelength of 540 nm in accordance with predetermined timing from the TG 37 and supplies the image signals to the image processing unit 51. The image processing unit 51 includes a color conversion processing section 51a for the narrow band light observation mode.

The operator can set the endoscope apparatus 1 to the narrow band light observation mode to cause the relatively thick blood vessel in the depth of the mucous membrane to be displayed on a screen 5a of the observation monitor 5 as shown in FIG. 3 in, for example, red color or magenta color with relatively high contrast.

Furthermore, the operator can also set the endoscope apparatus 1 to the narrow band light observation mode to cause not only the blood vessel below the surface of a living tissue but also a bleeding point at which bleeding has occurred to be drawn on the observation monitor 5. This is because even when bleeding occurs from the bleeding point on the mucous membrane surface of the mucous membrane and the mucous membrane surface is covered with the blood, when the blood is observed in the narrow band light observation mode, narrow band light in the vicinity of a wavelength of 600 nm passes through the blood and the blood running from the bleeding point on the mucous membrane surface is displayed on the observation monitor 5. Since a variation in a density (that is, concentration) of the blood flowing from the bleeding point or a variation in the thickness of the blood layer is high in the vicinity of the bleeding point, the flow of the blood flowing from the bleeding point is visualized so that the operator can visually recognize the blood flow, identify the bleeding point below the blood and the operator can speedily apply hemostasis treatment to the bleeding point.

Therefore, the color conversion processing section 51a of the image processing unit 51 in FIG. 1 assigns the respective image signals to respective channels of RGB of the observation monitor 5 and supplies the image signals to the selector 52. As a result, the relatively thick blood vessel 64 in the depth of the mucous membrane and the bleeding point at which bleeding has occurred are displayed on the screen 5a of the observation monitor 5 with high contrast.

For example, in order for the color conversion processing section 51a to display a blood vessel 64 in the depth with high contrast using narrow band light NL1 in the vicinity of a wavelength of 600 nm, the color conversion processing section 51a assigns the first image signal P1 (λ1), the second image signal P2 (λ2) and the third image signal P3 (λ3) to the G, R and B channels respectively.

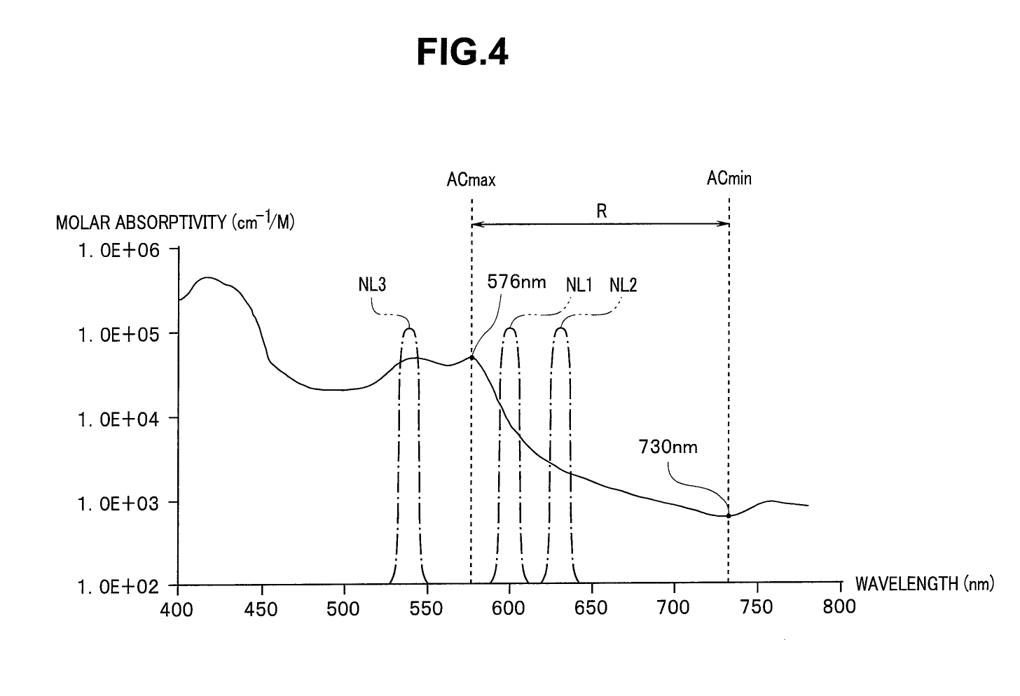

Here, light absorption characteristics of venous blood will be described. FIG. 4 is a diagram illustrating light absorption characteristics of venous blood. The vertical axis in FIG. 4 shows molar absorptivity (cm⁻¹/M) and the horizontal axis shows wavelength. Note that although three narrow band illuminating light beams are affected by scattering characteristics of the living tissue itself, the scattering characteristics of the living tissue itself monotonously decrease as the wavelength increases, and therefore FIG. 4 will be described as the absorption characteristics of the living tissue.

Generally, the venous blood contains oxygenated hemoglobin (HbO₂) and reduced hemoglobin (Hb) (hereinafter, both will be simply jointly referred to as “hemoglobin”) at a proportion of 60:40. Light is absorbed by hemoglobin, but the absorption coefficient thereof varies from one wavelength of light to another. FIG. 4 shows light absorption characteristics of venous blood for each wavelength from 400 nm to approximately 800 nm, and the absorptivity shows a maximum value at a point of wavelength of approximately 576 nm and a minimum value at a point of wavelength of 730 nm in a range from 550 nm to 750 nm.

In the narrow band light observation mode, three narrow band light beams are radiated and their respective returning light beams are received by the CCD 2.

The narrow band light in the vicinity of a wavelength of 600 nm (hereinafter referred to as “first narrow band light NL1”) is light in a wavelength band within a wavelength band R from a maximum value ACmax (here, absorptivity at a wavelength of 576 nm) to a minimum value ACmin (here, absorptivity at a wavelength of 730 nm) of absorption characteristics of hemoglobin.

The narrow band light in the vicinity of a wavelength of 630 nm (hereinafter, also referred to as “second narrow band light NL2”) is also light within the wavelength band R from the maximum value ACmax to the minimum value ACmin of absorption characteristics of hemoglobin, but it is light in a wavelength band having a longer wavelength than the first narrow band light NL1, lower absorptivity and with suppressed scattering characteristics of a living tissue. The suppressed scattering characteristics mean that the scattering coefficient decreases toward the long wavelength side.

That is, the light source apparatus 4 radiates first illuminating light NL1 having a peak wavelength in spectral characteristics between the wavelength band including the maximum value ACmax and the wavelength band including the minimum value ACmin in the absorption characteristics of the living tissue.

Furthermore, the light source apparatus 4 also radiates second illuminating light NL2 having lower absorption characteristic values than the image signal P1 resulting from the first illuminating light NL1 and having a peak wavelength in spectral characteristics with suppressed scattering characteristics of the living tissue.

Moreover, the light source apparatus 4 also radiates narrow band light in the vicinity of a wavelength of 540 nm (hereinafter, referred to as “third narrow band light NL3”). The third narrow band light NL3 is light in a wavelength band other than the wavelength band R from the maximum value ACmax to the minimum value ACmin in the absorption characteristics of hemoglobin and is illuminating light transmittable by a predetermined distance from the surface layer portion of the mucous membrane surface of the subject.

The CCD 2 outputs image pickup signals of the respective images of three narrow band light beams. Thus, each image includes a plurality of pixel signals based on respective returning light beams of the first, second and third narrow band light beams NL1, NL2 and NL3.

The first narrow band light NL1 and the second narrow band light NL2 repeat multiple scattering processes in the living tissue respectively, and are consequently emitted from the mucous membrane surface as returning light. The first narrow band light NL1 and the second narrow band light NL2 have their respective mean free paths. The mean free path of the first narrow band light NLI is shorter than the mean free path of the second narrow band light NL2.

Thus, the first narrow band light NL1 in the vicinity of a wavelength of 600 nm (λ1) reaches the vicinity of the blood vessel 64 and the second narrow band light NL2 in the vicinity of a wavelength of 630 nm (λ2) reaches a position slightly deeper than the blood vessel 64. Using this first narrow band light NL1 thereby makes it possible to display a relatively thick blood vessel having a diameter of 1 to 2 mm and a bleeding point at which bleeding has occurred, located in a relatively deep part, 1 to 2 mm below the surface layer of the mucous membrane of the living body.

The second narrow band light NL2 in the vicinity of a wavelength of 630 nm (λ2) also makes it possible to display a thicker blood vessel and a bleeding point at which bleeding has occurred, located in a deeper part.

Here, although the narrow band light NL1 or NL2 is light in the aforementioned wavelength band, the range of light in which the relatively thick blood vessel can be displayed with high contrast is from 585 nm which is the minimum wavelength to 630 nm which is the maximum wavelength.

The endoscope apparatus 1 radiates the above-described narrow band light, and can thereby cause the observation monitor 5 to display the blood vessel in the living tissue and the bleeding point at which bleeding has occurred.

As described above, the operator can use the aforementioned endoscope apparatus 1 to irradiate the subject with white light, irradiate the subject with narrow band light having a predetermined peak wavelength or switch from irradiation with white light to irradiation with narrow band light. The narrow band light is light in a red band of a visible range and including narrow band light having a peak wavelength in spectral characteristics between the wavelength band including a maximum value and the wavelength band including a minimum value in the hemoglobin light absorption characteristics of the living tissue of the subject.

### 3. Flow of Endoscopic Treatment of ESD

Next, an example of the method for endoscopic treatment of the present embodiment that applies treatment to a subject under an endoscope will be described.

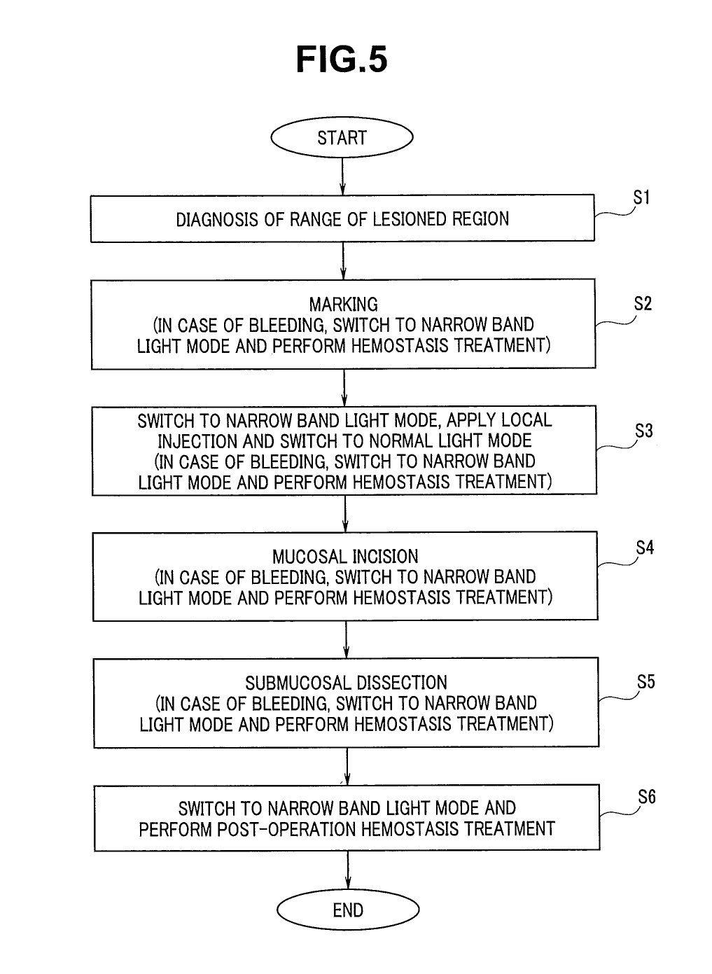

FIG. 5 is a flowchart illustrating a flow example of the method for endoscopic treatment in ESD. ESD is performed on the stomach, duodenum, esophagus, large intestine or the like. The method for endoscopic treatment of the present embodiment will be described below on a step-by-step basis.

### [Diagnosis of Range of Lesioned Region]

The operator inserts the endoscope 3 into the body of the subject by setting the observation mode which is one of operating modes of the endoscope apparatus 1 to a normal light observation mode and causes the distal end portion of the insertion portion 3a to approach the vicinity of the lesioned region by operating the bending portion while watching the image on the observation monitor 5 under white light observation.

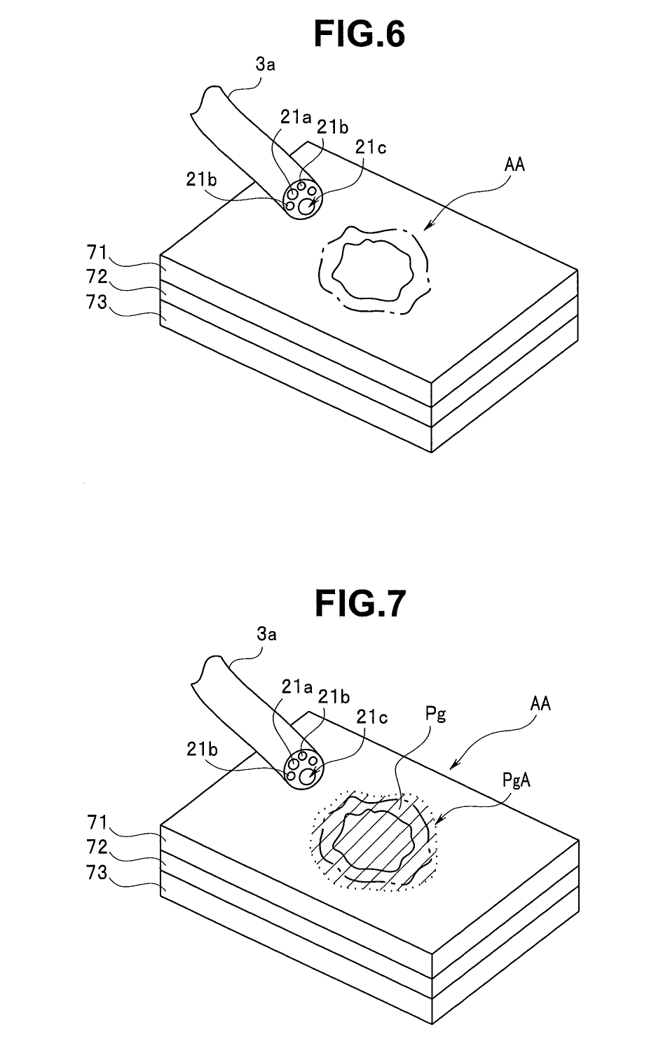

Upon visually recognizing the lesioned region by irradiating the subject with white light, the operator diagnoses the range of the lesioned region first (S1). FIG. 6 and FIG. 7 are diagrams illustrating a diagnosis of a range of a lesioned region. FIG. 6 is a diagram illustrating a situation in which the distal end portion of the insertion portion 3a of the endoscope 3 is brought close to a lesioned region AA and the lesioned region AA is included in the range of field of view of the endoscope 3. Note that in FIG. 6 and subsequent diagrams, a living tissue made up of a mucous membrane layer 71, a submucosa 72 and a muscular layer 73 is represented by a partially cut out rectangular parallelepiped.

The lesioned region AA is located in the mucous membrane layer 71, the submucosa 72 is located below the mucous membrane layer 71 and the muscular layer 73 is located below the submucosa 72. The observation mode of the endoscope apparatus 1 is a normal light observation mode and the operator places the distal end portion of the endoscope 3 in the vicinity of the lesioned region AA under white light observation.

The boundary between the lesioned region AA and a normal mucous membrane may be obscure as shown by a single-dot dashed line in FIG. 6 and FIG. 7. The operator therefore sprays a pigment Pg over the surface of the lesioned region AA. FIG. 7 is a diagram illustrating a situation in which the pigment Pg has been sprayed over the surface of the lesioned region AA. In FIG. 7, the diagonally shaded range shows a spray range PgA in which the pigment Pg has been sprayed.

The operator sprays the pigment Pg from the distal end portion of the insertion portion of the endoscope 3 via the forceps channel provided in the insertion portion 3a of the endoscope 3 over the lesioned region AA. The pigment Pg is indigo carmine or indigo carmine and acetic acid. The spray of the pigment Pg allows the operator to clearly view the boundary between the lesioned region AA and the normal mucous membrane displayed on the observation monitor 5. Since the boundary of the lesioned region AA is made clear by the pigment Pg, the operator can more easily perform marking treatment which will be described below.

In the above-described normal light observation mode, the operator makes a diagnosis of the range of the lesioned region AA.

Note that when the endoscope apparatus 1 has other observation modes other than the aforementioned normal light observation mode and narrow band light observation mode, the diagnosis of the range of the lesioned region may be made not in the normal light observation mode but in other observation modes. An example of the other observation modes is another narrow band light observation mode using narrow band light having a center wavelength of 415 nm or 540 nm.

### [Marking]

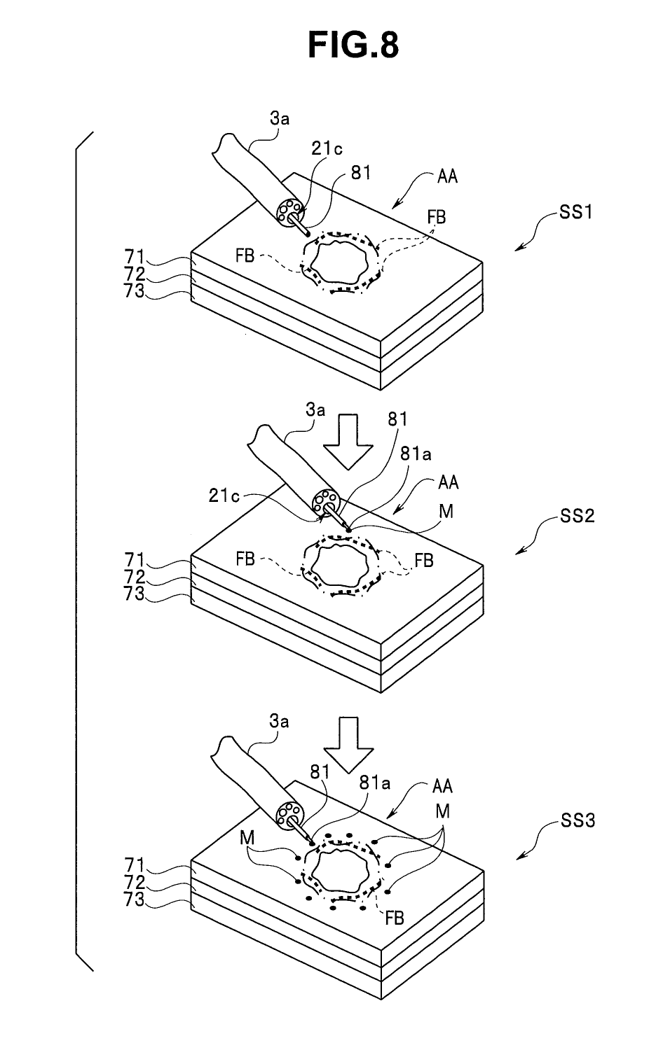

Next, the operator performs marking (S2). FIG. 8 is a diagram illustrating a marking example.

The operator performs marking after switching the observation mode of the endoscope apparatus 1 from the normal light observation mode to the narrow band light observation mode. That is, the operator applies marking treatment to the living tissue of the subject after irradiation with white light in S1. The marking is performed under narrow band light observation.

As described above, under narrow band light observation, a relatively thick blood vessel in the depth of the mucous membrane (hereinafter, also referred to as “deep blood vessel”) is displayed on the observation monitor 5 in a visually recognizable manner. Therefore, marking is performed so as to surround the whole circumference of the lesioned region AA at parts several mm away from the boundary of the lesioned region AA while avoiding the deep blood vessel FB shown by a thick dotted line in FIG. 8 using a high-frequency scalpel 81.

To be more specific, marking is performed by inserting the high-frequency scalpel 81 into the forceps channel of the insertion portion 3a, bringing the distal end portion of the insertion portion 3a close to the lesioned region AA as shown in SS1 in FIG. 8, and causing a distal end portion 81a of the high-frequency scalpel 81 to eject from a treatment opening of the distal end portion of the insertion portion 3a and contact the surface of the living tissue as shown in SS2.

As shown in SS2, when the high-frequency scalpel 81 is made to eject from the treatment instrument opening 21c of the distal end portion of the insertion portion 3a and the distal end portion 81a is made to contact the mucous membrane layer 71, a mark M is created. During the marking, a plurality of marks M are created on the surface of the living tissue around the deep blood vessel so as to surround the whole circumference of the lesioned region AA as shown in SS3 in FIG. 8.

When the marking is finished, the operator switches the observation mode of the endoscope apparatus 1 from the narrow band light observation mode to the normal light observation mode and checks the presence or absence of bleeding in the normal light observation mode.

If bleeding is detected during the marking, the operator keeps the narrow band light observation mode as the observation mode and checks the bleeding point and performs hemostasis treatment.

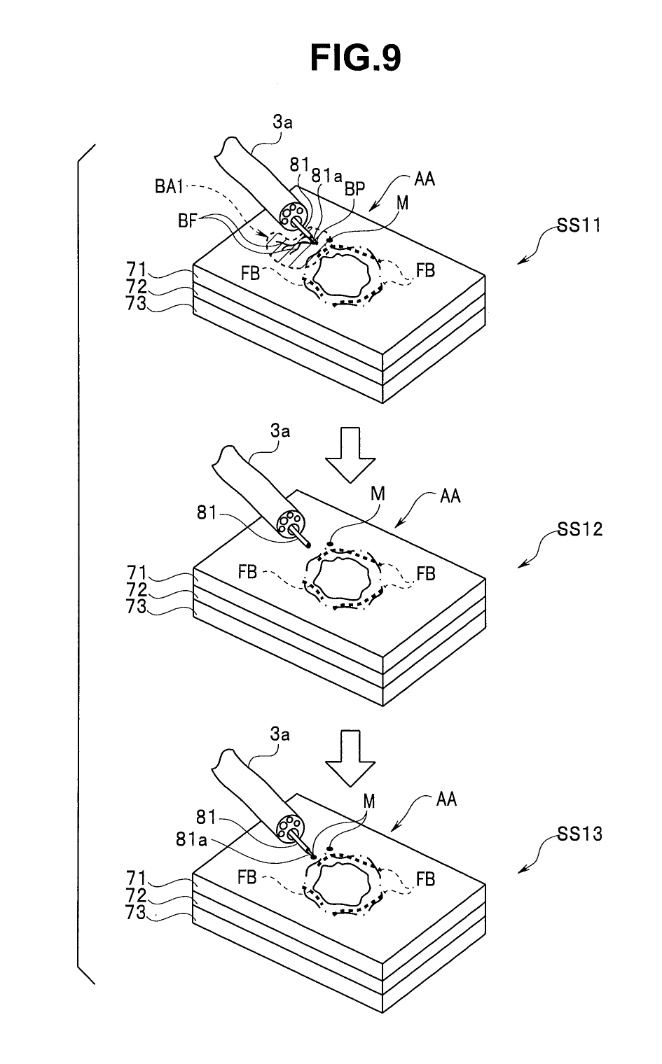

FIG. 9 is a diagram illustrating hemostasis treatment when bleeding occurs during the marking. After the bleeding is detected, the operator can visually recognize the bleeding point from the observation monitor 5 in the narrow band light observation mode, and can thereby recognize a bleeding flow BF in a bleeding region BA1 shown by the oblique lines and identify a bleeding point BP from the flow BF as shown in SS11.

As a result, the operator can perform hemostasis treatment using the high-frequency scalpel 81 or hemostasis forceps. In the case of the high-frequency scalpel 81, hemostasis treatment is performed by making the distal end portion 81a of the high-frequency scalpel 81 contact the bleeding point BP and passing a high-frequency current therethrough. In the case of the hemostasis forceps, hemostasis treatment is performed by grasping the bleeding point BP with the surface of the grasping portion of the distal end of the hemostasis forceps and passing a high-frequency current therethrough.

After the hemostasis treatment, the operator switches the observation mode from the narrow band light observation mode to the normal light observation mode to thereby switch from irradiation with narrow band light to irradiation with white light and checks whether or not the hemostasis treatment has been completed as shown in SS12.

Upon confirming that the hemostasis treatment has been completed, the operator switches the observation mode from the normal light observation mode to the narrow band light observation mode and continues marking treatment as shown in SS13.

As described above, during marking, by radiating narrow band light having a predetermined peak wavelength, the operator can check the bleeding point and perform hemostasis treatment. After the hemostasis, the operator switches from the narrow band light observation mode to the normal light observation mode and confirms that the hemostasis treatment has been completed.

### [Local Injection]

Returning to FIG. 5, the operator switches the observation mode from the normal light observation mode to the narrow band light observation mode, performs local injection, and then switches the observation mode to the normal light observation mode (S3). That is, the operator performs local injection treatment on the living tissue of the subject after irradiation with white light. The local injection is performed under narrow band light observation.

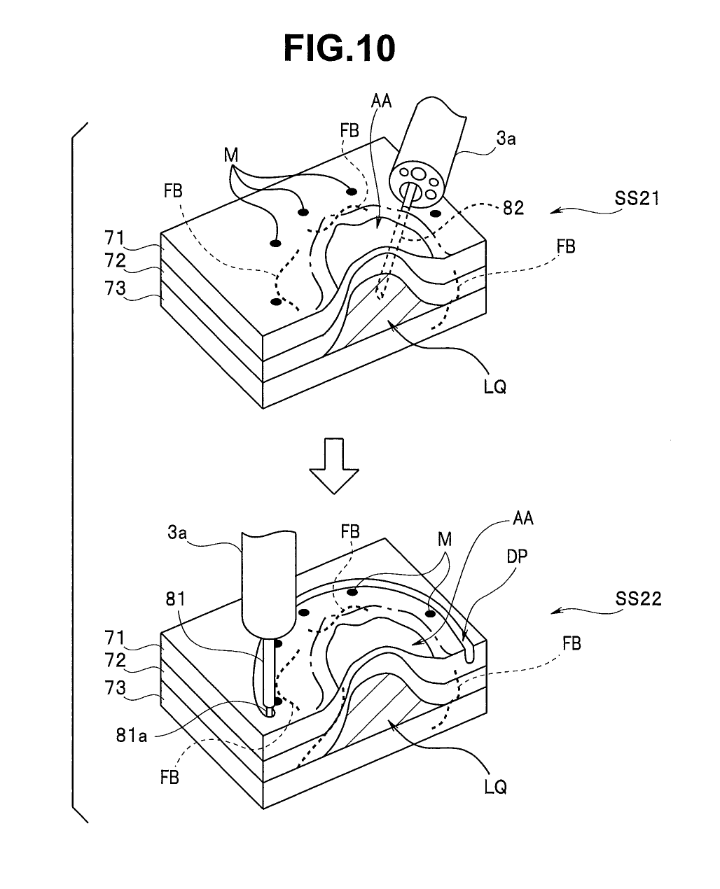

FIG. 10 is a diagram illustrating treatment from local injection to mucosal incision. SS21 in FIG. 10 is a diagram illustrating the local injection.

When the observation mode is switched to the narrow band light observation mode, the relatively thick blood vessel in the depth of the mucous membrane is displayed in, for example, red color or magenta color. Thus, the operator passes a local injection needle 82 through the forceps channel, and can thereby inject a local injection liquid LQ (shown by the oblique lines) into the submucosa 72 while avoiding the deep blood vessel FB (shown by a dotted line) which is visually recognizable in the narrow band light observation mode as shown in SS21 in FIG. 10.

When bleeding occurs during the local injection, the operator must perform hemostasis and the operation time is extended by the hemostasis time. Thus, the operator can perform local injection while avoiding the deep blood vessel, and can thereby prevent bleeding during the operation and shorten the operation time. After that, the operator removes the local injection needle 82, switches the observation mode to the normal light observation mode to thereby switch from the irradiation with narrow band light to irradiation with the white light and checks the presence or absence of bleeding.

Note that if the blood vessel is punctured by the distal end of the local injection needle 82 and bleeding occurs during the local injection, as shown in SS11 and SS12 in FIG. 9, the operator performs hemostasis treatment in the narrow band light observation mode. After the hemostasis treatment, the operator switches from the narrow band light observation mode to the normal light observation mode and confirms that the hemostasis treatment has been completed.

Note that local injection is performed as appropriate also in treatment of mucosal incision and submucosal dissection as will be described later.

As described above, in local injection, by switching from irradiation with the white light to the irradiation with narrow band light having a predetermined peak wavelength according to the condition of bleeding that occurs in the living tissue and radiating narrow band light, the operator can perform hemostasis treatment while confirming the bleeding point.

### [Mucosal Incision]

After local injection, the operator sets the observation mode to the normal light observation mode and performs mucosal incision (S4). With the high-frequency scalpel 81 inserted through the forceps channel, the operator performs mucosal incision as shown in SS22 in FIG. 10. That is, the operator performs mucosal incision treatment on the living tissue of the subject after irradiation with the white light.

The mucosal incision is performed by causing the distal end portion 81a of the high-frequency scalpel 81 to contact the living tissue outside the marks M created by marking. By moving the distal end portion 81a along the outer circumferential portion within the range surrounded by the plurality of marks M, the operator can form a notch as shown in SS22 in FIG. 10. An incision portion DP which is the notch formed by the distal end portion 81a of the high-frequency scalpel 81 is formed outside the plurality of marks M created by marking. SS22 in FIG. 10 shows a situation in which the incision portion DP has been formed up to a midpoint of the outer circumferential portion within the range surrounded by the plurality of marks M.

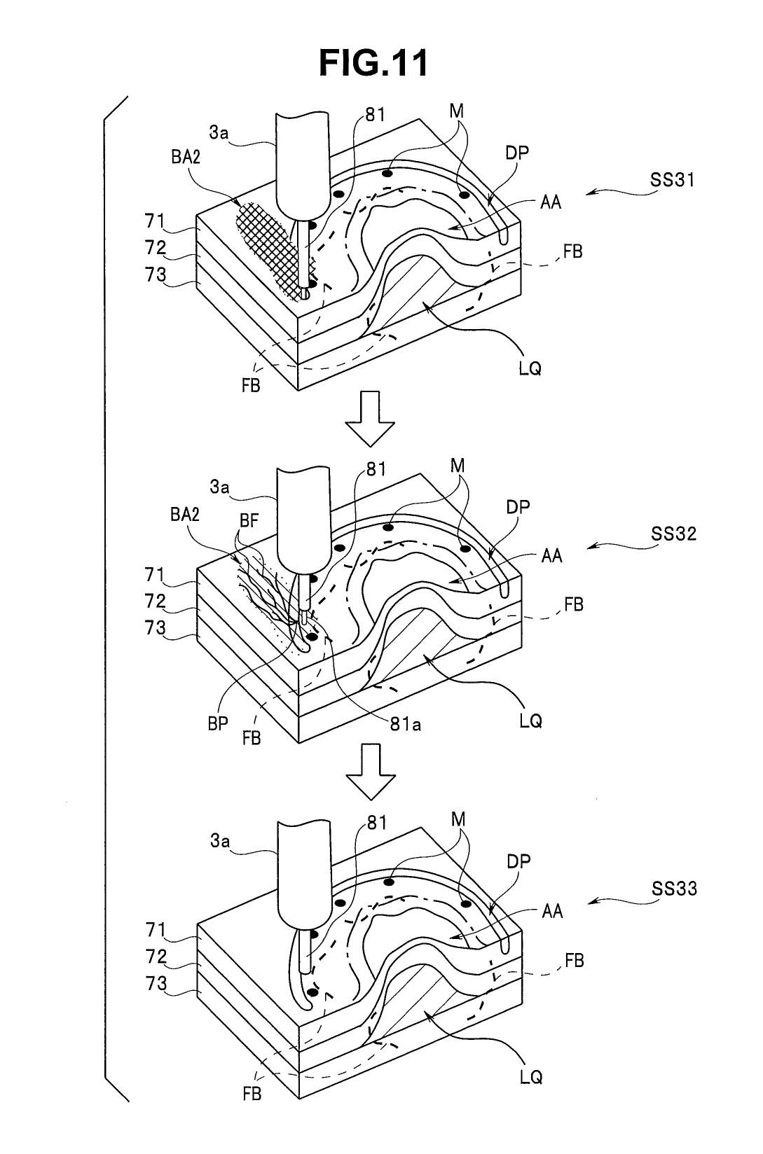

If bleeding occurs during the mucosal incision, the operator switches the observation mode from the normal light observation mode to the narrow band light observation mode and performs hemostasis treatment. FIG. 11 is a diagram illustrating hemostasis treatment when bleeding occurs during mucosal incision.

For example, as shown in SS31 in FIG. 11, when the operator damages the deep blood vessel FB (shown by a dotted line) by the distal end portion 81a of the high-frequency scalpel 81 during the mucosal incision and bleeding occurs, the operator cannot check the bleeding point below a bleeding region BA2 shown by a shaded area under normal observation.

Thus, the operator switches the observation mode to the narrow band light observation mode so as to visually recognize the bleeding point BP from a bleeding flow BF that exists below the bleeding region BA2 shown by the shaded area as shown in SS32. Upon detecting the bleeding point BP, the operator performs hemostasis using the high-frequency scalpel 81 (or hemostasis forceps). In the narrow band light observation mode, the bleeding region BA2 is displayed, for example, in yellow color or orange color, the bleeding flow BF is displayed in dark orange color, the bleeding point BP is displayed in yellow color and the deep blood vessel FB is displayed, for example, in red color or magenta color.

After the hemostasis treatment, the operator switches the observation mode to the normal light observation mode to thereby switch from irradiation with narrow band light to irradiation with white light and confirms that the hemostasis treatment has been completed as shown in SS33.

That is, the operator can perform hemostasis treatment while checking the bleeding point by switching irradiation with white light to irradiation with narrow band light having a predetermined peak wavelength in accordance with the condition of bleeding that occurs from the living tissue and radiating narrow band light during mucosal incision.

After the hemostasis, the operator resumes mucosal incision in the normal light observation mode, performs mucosal incision around the whole circumference of the lesioned region AA and then shifts to submucosal dissection treatment.

If no bleeding occurs during mucosal incision, the observation mode is kept to the normal light observation mode and not shifted to the narrow band light observation mode.

### [Submucosal Dissection]

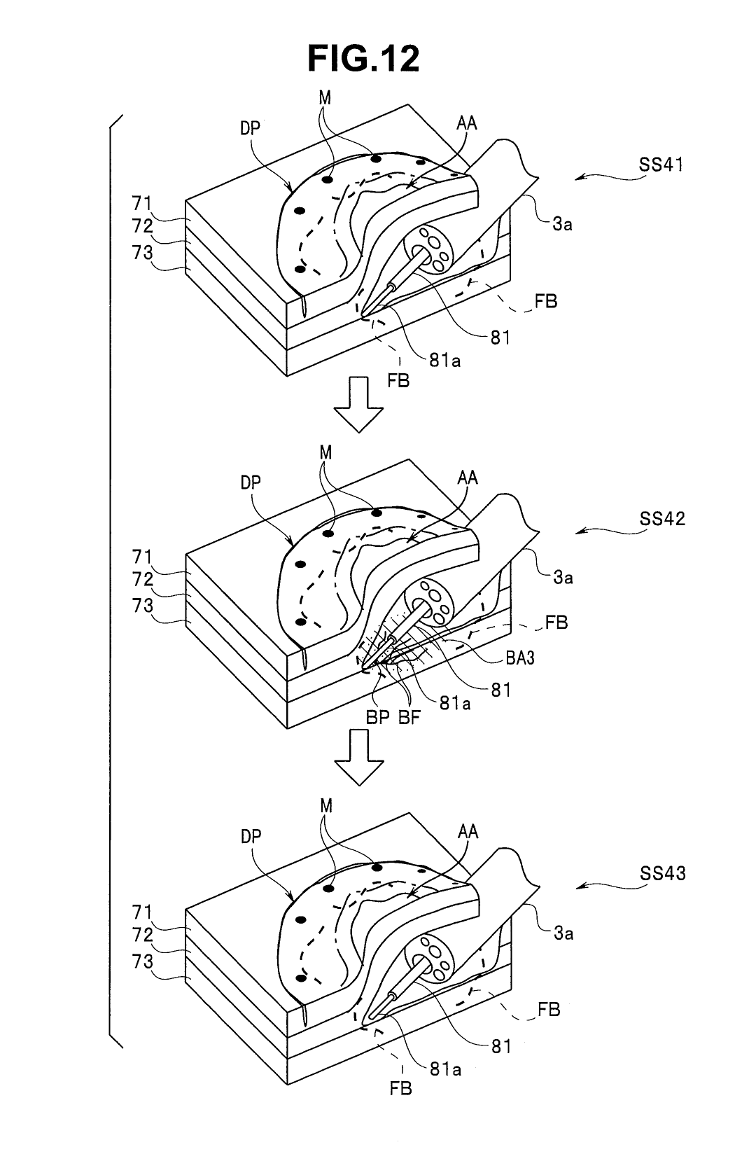

Returning to FIG. 5, the operator performs treatment of submucosal dissection next (S5). Submucosal dissection is performed using the high-frequency scalpel 81 in the normal light observation mode. That is, the operator performs submucosal dissection treatment on the living tissue of the subject after irradiation with white light. FIG. 12 is a diagram illustrating submucosal dissection treatment. As shown in SS41, submucosal dissection is performed by causing the distal end portion 81a of the high-frequency scalpel 81 to contact the submucosa 72 directly on the muscular layer 73.

If bleeding occurs during the submucosal dissection, the observation mode is switched from the normal light observation mode to the narrow band light observation mode and hemostasis treatment is performed. As shown in SS42 in FIG. 12, if the operator damages the deep blood vessel FB by the distal end portion 81a of the high-frequency scalpel 81 during the submucosal dissection and bleeding occurs, the operator cannot check the bleeding point below a bleeding region BA3 under normal observation.

Thus, the operator switches the observation mode to the narrow band light observation mode so as to visually recognize the bleeding point BP located below the region BA3 shown by the oblique lines as shown in SS42. Upon detecting the bleeding point BP from the bleeding flow BF, the operator performs hemostasis using the high-frequency scalpel 81 (or hemostasis forceps).

After the hemostasis treatment, the operator switches the observation mode to the normal light observation mode to thereby switch from irradiation with narrow band light to irradiation with white light, confirms that the hemostasis treatment has been completed as shown in SS43 and resumes submucosal dissection in the normal light observation mode.

That is, in submucosal dissection, the operator performs hemostasis treatment after checking the bleeding point by switching from irradiation with white light to irradiation with narrow band light having a predetermined peak wavelength according to the condition of bleeding that occurs in the living tissue and radiating narrow band light.

If no bleeding occurs during submucosal dissection, the observation mode is kept to the normal light observation mode and not shifted to the narrow band light observation mode.

### [Post-Operation Hemostasis]

The operator then performs post-operation hemostasis treatment (S6). Upon completion of the submucosal dissection treatment, the operator switches the observation mode from the normal light observation mode to the narrow band light observation mode, checks the deep blood vessel FB located in the vicinity of an incision surface DS and coagulates the deep blood vessel FB located in the vicinity of the incision surface DS using the high-frequency scalpel 81. That is, after the submucosal dissection treatment, the operator performs preventive hemostasis treatment on the living tissue while radiating narrow band light.

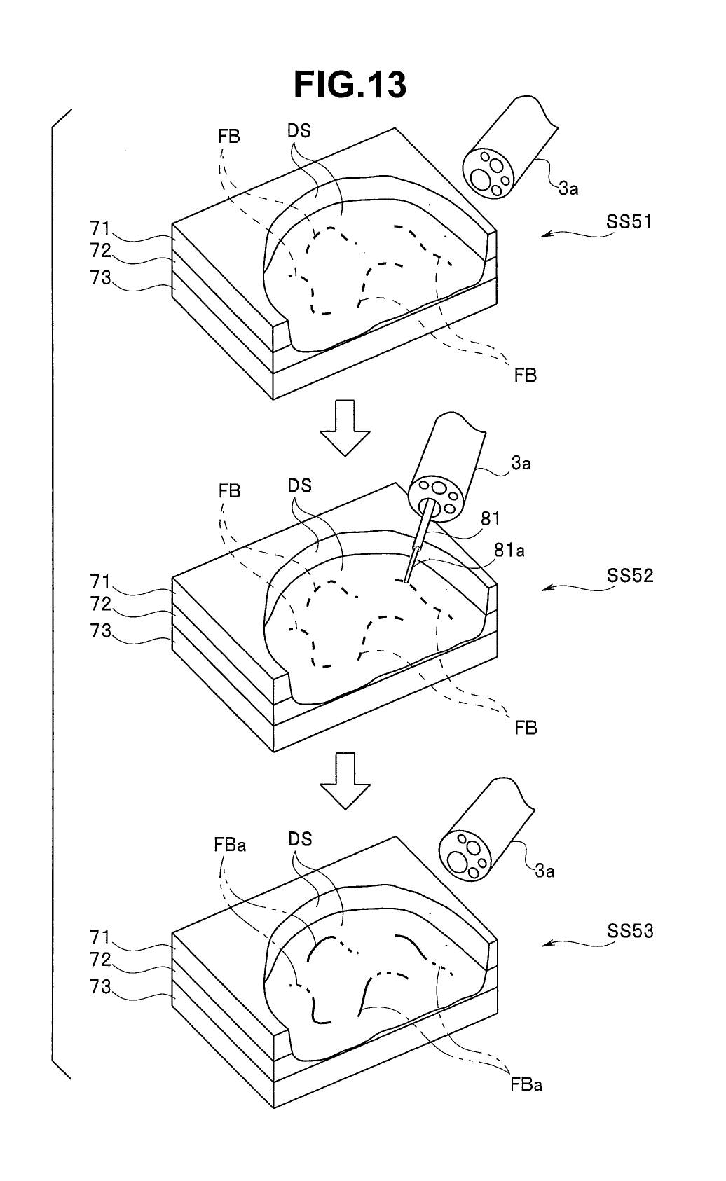

FIG. 13 is a diagram illustrating the post-operation hemostasis treatment. In SS51 and SS52 in FIG. 13, the deep blood vessel FB located in the vicinity of the incision surface DS is shown by a dotted line. The deep blood vessel FB is located in the vicinity of the incision surface DS of the portion from which the mucous membrane layer 71 and the submucosa 72 have been removed by submucosal dissection.

Since the incision surface DS by submucosal dissection treatment appears in red color under white color normal light observation, it is difficult for the operator to visually recognize the deep blood vessel FB beneath the incision surface DS. After the operation, since bleeding is likely to occur from the deep blood vessel FB beneath the incision surface DS, it is desirable to coagulate the deep blood vessel FB located in the vicinity of the surface of the incision surface DS.

After the submucosal dissection treatment, the operator switches the observation mode to the narrow band light observation mode and causes the observation monitor 5 to display the deep blood vessel FB located in the vicinity of the incision surface DS in red color or magenta color to check the position of the deep blood vessel FB.

Next, the operator causes the distal end portion 81a of the high-frequency scalpel 81 to contact the incision surface DS on the detected deep blood vessel FB or causes the detected thick blood vessel FB to be grasped with the surface of the grasping portion at the distal end of the hemostasis forceps.

As shown in SS52, by passing a high-frequency current through the distal end portion 81a of the high-frequency scalpel 81 or the distal end of the hemostasis forceps, the operator can cause the deep blood vessel FB in the vicinity of the incision surface DS to coagulate and perform preventive hemostasis. Post-operation hemostasis treatment is applied to the whole deep blood vessel FB located in the vicinity of the incision surface DS. As shown in SS53 in FIG. 13, the deep blood vessel FB subjected to the coagulation treatment is shown by a two-dot dashed line.

Upon completion of the coagulation treatment on the deep blood vessel FB, the operator switches the observation mode from the narrow band light observation mode to the normal light observation mode, observes the whole treatment region as shown in SS53, makes sure that there is no bleeding and removes, when there is no bleeding, the insertion portion 3a of the endoscope 3 from the inside of the body.

As described above, according to the aforementioned embodiment, when bleeding occurs during ESD in the stomach or the like, the operator can speedily perform hemostasis treatment.

### (Treatment Other Than ESD)

Note that in the aforementioned ESD in the stomach or the like, when bleeding occurs in respective treatments from marking to submucosal dissection, the observation mode is switched to the narrow band light observation mode so as to detect the bleeding point and then hemostasis treatment is performed. However, the aforementioned procedure is also applicable to EMR (endoscopic mucosal resection) and EST (endoscopic sphincterotomy). Even when bleeding occurs in EMR or EST, the operator can likewise switch the observation mode to the narrow band light observation mode and check the bleeding point before performing hemostasis treatment.

Moreover, the aforementioned procedure is also applicable to a case where bleeding occurs when treatment under an endoscope is performed on other organs such as a liver and a kidney. Since an organ such as the liver contains many blood vessels, oozing bleeding frequently occurs, and employing the aforementioned narrow band observation light mode in such a case allows the operator to visually recognize the bleeding point and speedily perform hemostasis treatment.

Also in various brain surgery-related treatments, the aforementioned procedure is also applicable when bleeding occurs during treatment. Hemostasis during brain surgery is generally performed by dressing over the bleeding location with gauze without using an electric knife or the like, but by switching the observation mode to the narrow band light observation mode in case of massive bleeding, the operator can check the location to dress with gauze using an image in the narrow band light observation mode and thereby speedily perform hemostasis treatment.

Furthermore, even when pulsatile bleeding occurs during treatment, the bleeding occupies 50% or more of the field of view, but by switching the observation mode to the narrow band light observation mode in case of pulsatile bleeding, the operator can check the bleeding point, and thereby speedily perform hemostasis treatment.

When pulsatile bleeding occurs during radiation of normal light which is white light, the operator switches the observation mode to the narrow band light observation mode, and selects irradiation with narrow band light having a peak wavelength in spectral characteristics in a red band of the visible range between a wavelength band including a maximum value and a wavelength band including a minimum value in hemoglobin light absorption characteristics of the living tissue of the subject. When pulsatile bleeding occurs, this allows the operator to visually recognize the bleeding point without performing forward water feeding into the bleeding region from the distal end portion of the insertion portion of the endoscope, and thereby speedily perform hemostasis treatment.

As described above, when bleeding occurs during treatment, it is conventionally not easy to check the position of the bleeding point beneath the blood, whereas according to the aforementioned embodiment, when bleeding occurs during treatment, the operator can easily check the bleeding point under the blood, and thereby speedily perform endoscopic treatment.

### Second Embodiment

Next, a second embodiment will be described. In the first embodiment, each treatment is performed during operation, the operator switches the observation mode to the narrow band light observation mode in accordance with the condition of bleeding during treatment, and can thereby check the bleeding point for hemostasis. On the other hand, in the second embodiment, the operator is allowed to check the condition of a blood flow in order to check the effect of treatment after the treatment.

Since an endoscope apparatus used for a method for endoscopic treatment according to the second embodiment is similar to the endoscope apparatus 1 described in the first embodiment, the description of the configuration of the apparatus is omitted and the method for endoscopic treatment of the second embodiment will be described. Hereinafter, treatment of cerebral aneurysm clipping and treatment of polypectomy of the large intestine will be mainly described.

### 1. Flow of Endoscopic Treatment of Cerebral Aneurysm Clipping

An example of the method for endoscopic treatment in cerebral aneurysm clipping according to the present embodiment will be described.

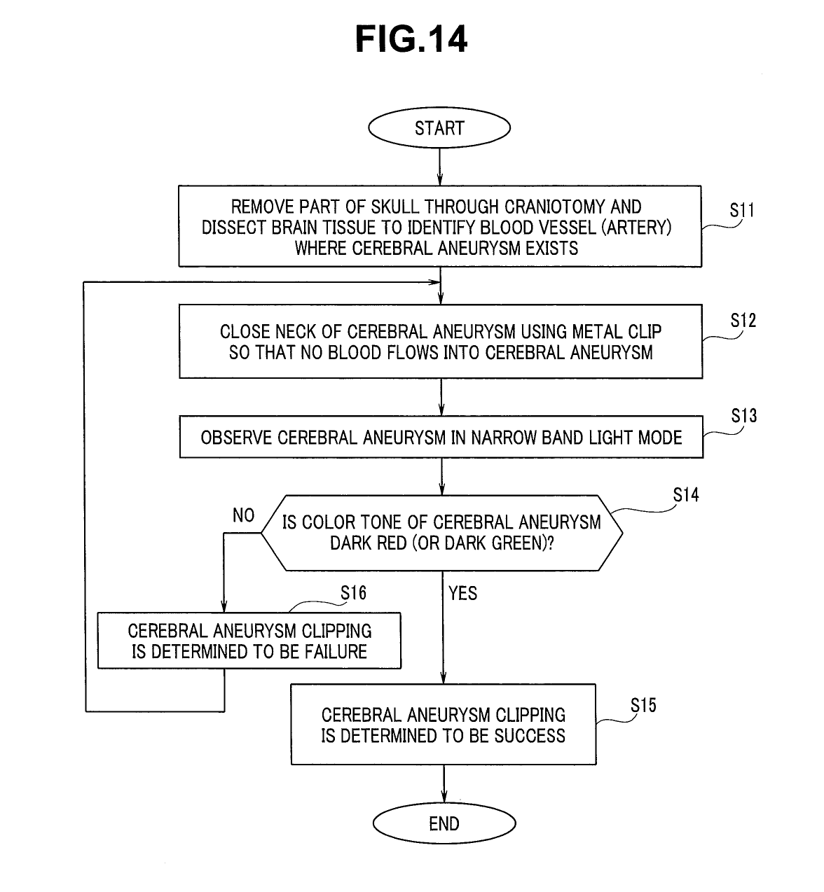

FIG. 14 is a flowchart illustrating a flow example of the method for endoscopic treatment in cerebral aneurysm clipping. Hereinafter, the method for endoscopic treatment according to the present embodiment will be described on a step-by-step basis.

### [Identification of Blood Vessel Where Cerebral Aneurysm Exists]

The operator removes a part of the skull through craniotomy on the subject and dissects the brain tissue to identify the artery which is the blood vessel where cerebral aneurysm exists (S11). Such identification of the artery in the brain is performed by setting the observation mode of the endoscope apparatus 1 to a normal light observation mode.

### [Closure by Clip]

Next, the operator closes the neck of the cerebral aneurysm using a metal clip so that no blood flows into the aneurysm (S12). That is, the treatment in S12 is performed on the living tissue of the subject after irradiating the subject with white light.

The neck of the cerebral aneurysm is a boundary portion between the cerebral aneurysm and a normal blood vessel.

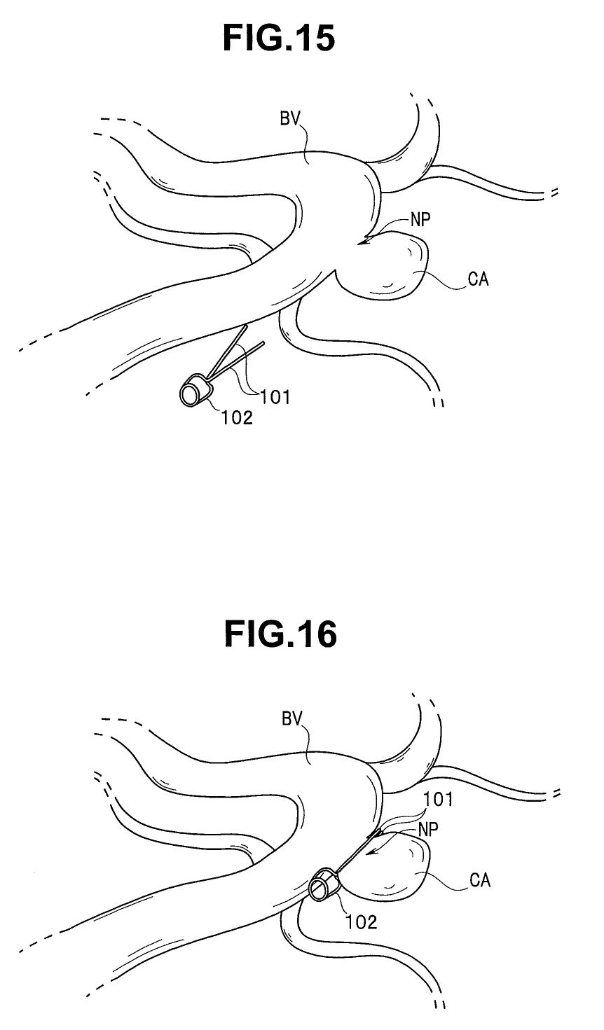

FIG. 15 and FIG. 16 are diagrams illustrating clipping applied to the neck of the cerebral aneurysm. FIG. 15 is a diagram illustrating a cerebral aneurysm CA which has developed in a blood vessel BV, and a clip CL. As shown in FIG. 15, the blood vessel BV is branched into a plurality of portions and the cerebral aneurysm CA has developed in part of the blood vessel BV.

The clip CL is metallic and has two arm-like blood vessel pinching portions 101 and a stem 102. The clip CL is grasped, for example, by clip forceps and attached to a neck NP of the cerebral aneurysm CA. FIG. 16 is a diagram illustrating a case where the clip CL has been correctly attached to the neck NP of the cerebral aneurysm CA. As shown in FIG. 16, when the clip CL is attached to the neck NP of the cerebral aneurysm CA, the neck NP is closed so as to prevent the blood from entering the aneurysm CA.

### [Observation of Aneurysm in Narrow Band Light Observation Mode]

After clipping the neck NP of the cerebral aneurysm CA, the operator switches the observation mode to the narrow band light observation mode and observes the aneurysm CA (S13). That is, after the clipping treatment, irradiation with white light is switched to irradiation of the subject with narrow band light having a predetermined peak wavelength.

As shown in FIG. 16, when the neck NP of the cerebral aneurysm CA is correctly clipped and closed, and the blood flow into the cerebral aneurysm CA is blocked, the blood in the cerebral aneurysm CA is not emphasized in the narrow band light observation mode and displayed in dark red or dark green.

### [Determination of Appropriateness of Clipping Treatment]

The operator determines whether the color tone of an image of the cerebral aneurysm CA in the narrow band light observation mode displayed on the observation monitor 5 is dark red or dark green (S14). As described above, when the neck NP of the cerebral aneurysm CA has been correctly clipped by the clip CL, the blood in the cerebral aneurysm CA is not emphasized in the narrow band light observation mode, and displayed in dark red or dark green. However, when the clip CL is not correctly clipping the neck NP of the cerebral aneurysm CA, the blood in the cerebral aneurysm CA is emphasized in the narrow band light observation mode and displayed in vivid red or vivid green.

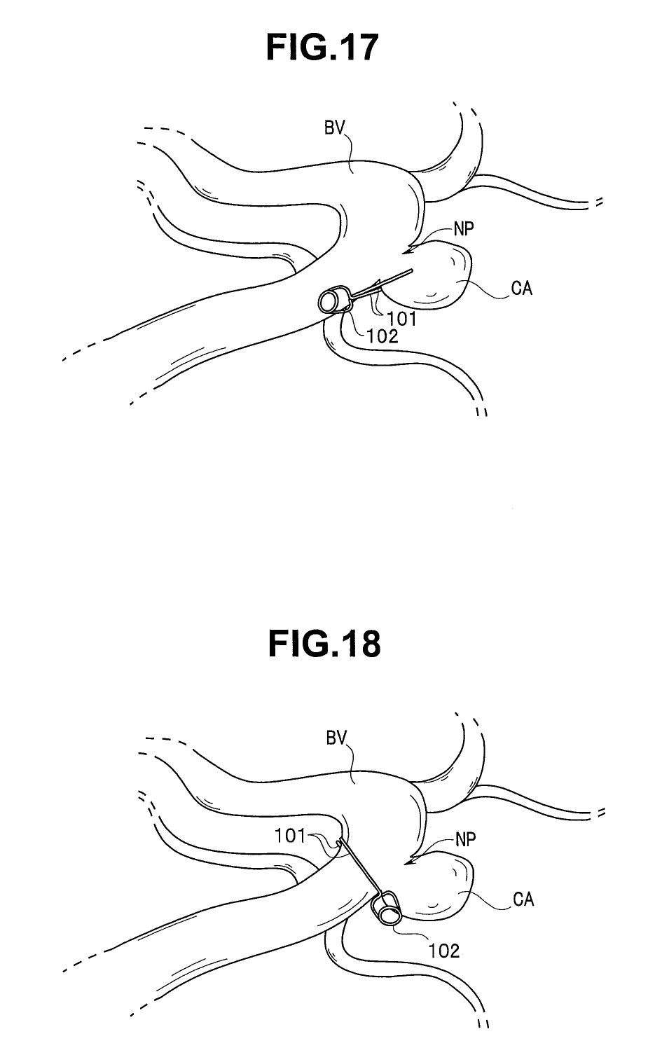

FIG. 17 and FIG. 18 are diagrams illustrating inappropriate clipping. FIG. 17 is a diagram illustrating a case where the clip CL is clipping only up to a midpoint of the neck NP of the cerebral aneurysm CA. In the case of FIG. 17, since the neck of the aneurysm CA is not completely clipped, the blood flows into the cerebral aneurysm CA and both the interior of the cerebral aneurysm CA and the blood vessel BV are emphasized in the narrow band light observation mode and displayed in vivid red or vivid green.

On the other hand, FIG. 18 is a diagram illustrating a situation in which the clip CL is clipping not the neck NP of the cerebral aneurysm CA but the blood vessel BV. In the case of FIG. 18, since the neck NP of the cerebral aneurysm CA is not completely clipped, the blood flows into the cerebral aneurysm CA, both the interior of the cerebral aneurysm CA and the blood vessel BV are emphasized in the narrow band light observation mode and displayed in vivid red or vivid green. Furthermore, the blood vessel BV on the downstream side of the blood flow is displayed in white.

Thus, when the color tone of the image of the cerebral aneurysm CA in the narrow band light observation mode is dark red or dark green (S14: YES), the operator can determine that cerebral aneurysm clipping has been successfully performed (S15) and finishes cerebral aneurysm clipping under craniotomy.

On the other hand, when the color tone of the image of the cerebral aneurysm CA in the narrow band light observation mode is not dark red or dark green (S14: NO), the operator can determine that the cerebral aneurysm clipping has failed (S16), removes the clip CL from the blood vessel BV, returns to S12 and performs clipping again.

Thus, after the treatment, the operator determines the color tone of the cerebral aneurysm CA in the narrow band light observation mode, thereby checks the condition of the blood flow after the treatment, and can determine whether or not appropriate clipping treatment has been performed.

As described above, according to the aforementioned embodiment, since it is possible to check whether or not the blood flow is in an inappropriate condition by the treatment, the operator can thereby speedily perform endoscopic treatment.

### 2. Flow of Endoscopic Treatment of Polypectomy of the Large Intestine

As another example, a method for endoscopic treatment of polypectomy of the large intestine will be described on a step-by-step basis.

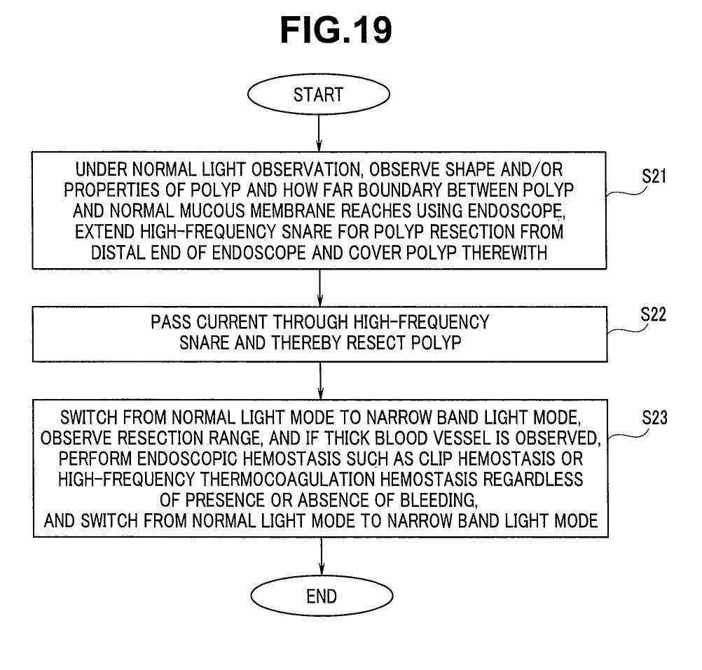

FIG. 19 is a flowchart illustrating a flow example of the method for endoscopic treatment in polypectomy of the large intestine.

### [Attachment of High-Frequency Snare]

The operator sets the observation mode to the normal light observation mode, observes the shape and/or properties of a polyp in the large intestine, and how far the boundary between the polyp and the normal mucous membrane reaches using the endoscope 3 under normal light observation, extends a high-frequency snare for polyp resection from the distal end of the endoscope 3 and covers the polyp therewith (S21).

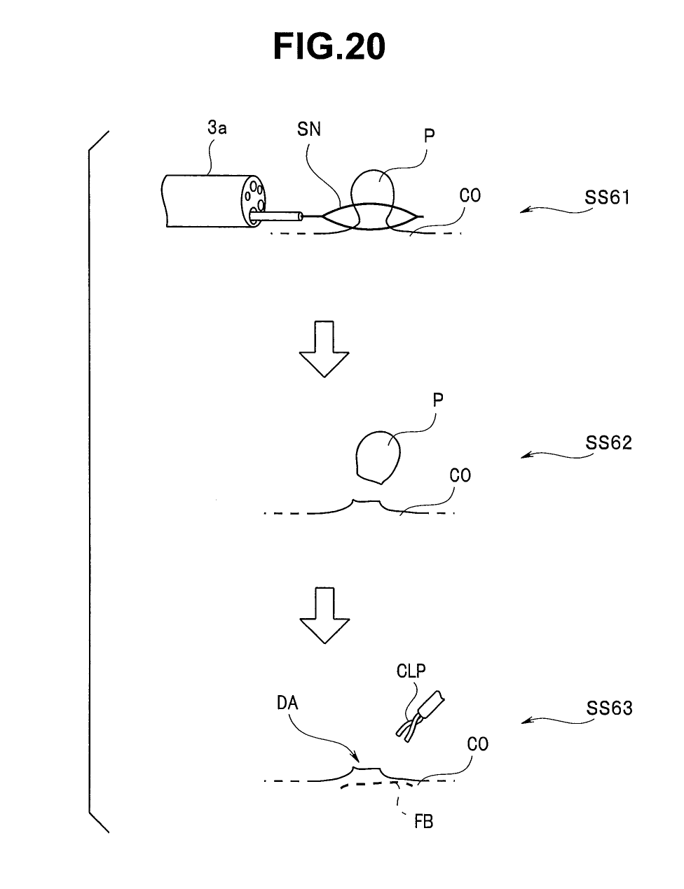

FIG. 20 is a diagram illustrating polypectomy of the large intestine. A polyp P is formed on the inner wall of a large intestine CO. As shown in SS61, the operator observes the polyp P and the periphery thereof under normal light observation and attaches a high-frequency snare SN so as to cover the polyp P. The high-frequency snare SN is a loop-shaped electric knife for polyp resection.

### [Polyp Resection]

Next, the operator passes a current through the high-frequency snare SN and thereby resects the polyp P (S22). As shown in SS62 in FIG. 20, the polyp P is resected from the mucous membrane of the large intestine.

### [Endoscopic Hemostasis Treatment]

After the resection of the polyp P, the operator switches the observation mode to the narrow band light observation mode, observes a resection range DA from which the polyp P has been resected, and if a thick deep blood vessel FB is observed, the operator performs endoscopic hemostasis such as clip hemostasis or high-frequency thermocoagulation hemostasis regardless of the presence or absence of bleeding, and switches from the normal light mode to the narrow band light mode (S23). That is, after the resection treatment of the polyp P, irradiation with white light is switched to irradiation of the subject with narrow band light having a predetermined peak wavelength.

As shown in SS63 in FIG. 20, the operator observes the resection range DA of the polyp P, and since the deep blood vessel FB is visible under narrow band light observation, when the thick deep blood vessel FB is observed, the operator performs endoscopic preventive hemostasis treatment. Clip hemostasis is treatment that pinches the thick blood vessel by a clip CLP and applies astriction thereto. Thermocoagulation hemostasis is performed by causing the distal end of a high-frequency device to contact the upper mucous membrane surface of the thick blood vessel and passing a current therethrough.

Note that instead of thermocoagulation hemostasis, clip hemostasis may also be performed. That is, after irradiation with narrow band light, the operator applies coagulation treatment or clip hemostasis to a non-bleeding blood vessel of the living tissue. After the coagulation treatment or clip hemostasis, irradiation with narrow band light is switched to irradiation with white light.

Thus, after the treatment, the operator observes the polyp P resection range DA in the narrow band light observation mode, and can perform endoscopic preventive hemostasis treatment.

As described above, according to the aforementioned embodiment, it is possible to perform preventive hemostasis treatment after checking the condition of the blood flow after the treatment, and thereby speedily perform endoscopic treatment.

The treatment of cerebral aneurysm clipping and the treatment of polypectomy of the large intestine have been described so far, and checking the condition of the blood flow is also applicable to other treatments to confirm effects of the treatment or perform preventive hemostasis treatment after the treatment.

For example, after treatment such as anastomosis after large intestine resection, segmental resection of a liver, kidney or lung, biliary excretion, urinary excretion or the like, effects of the respective treatments can be checked.

In the case of anastomosis treatment after large intestine resection, the observation mode is switched to the narrow band light observation mode, for example, to check whether or not vascular anastomosis has been appropriately performed, whether or not the blood is flowing into the blood vessel, that is, the condition of the blood flow.

After the anastomosis, if the blood flow can be detected, the operator can determine that the anastomosis has been performed appropriately.

In the case of segmental resection treatment of the liver, the operator applies clipping to the hepatic artery and resects the lesioned region while avoiding any blood flow around the lesioned region. However, if some blood flow remains in the resected region without being clipped, bleeding occurs on resecting. Therefore, to check effects of the clipping after the clipping, the operator switches the observation mode to the narrow band light observation mode so as to visually recognize the region where the blood flow is blocked by the clipping and the unclipped region where a blood flow exists.

Thus, when it is confirmed that the region including the lesioned region to be resected includes a region where a blood flow exists, the operator applies further clipping to the hepatic artery which is not clipped, and can thereby ensure that the region including the lesioned region to be resected does not include the region where a blood flow exists.

Furthermore, when observing the bile duct in gallstone treatment, the operator switches the observation mode to the narrow band light observation mode after biliary excretion from the bile duct, and can thereby identify the gallstones existing in the remaining bile.

Similarly, in ureterolith treatment, the operator switches the observation mode to the narrow band light observation mode after urinary excretion from the ureter, and can thereby identify ureteral stones remaining in the urine and transurethrally crush or extirpate the ureteral stones.

As described above, while the condition of a blood flow after treatment cannot be conventionally checked, according to the aforementioned second embodiment, it is possible to check the condition of the blood flow after the treatment and thereby speedily perform endoscopic treatment.

Note that although the methods for endoscopic treatments according to the aforementioned two embodiments use a so-called frame-sequential endoscope apparatus using a monochrome image pickup device, a so-called simultaneous endoscope apparatus using a three primary color image pickup device or a complementary color image pickup device may also be used. In the case of a simultaneous endoscope apparatus, a plurality of light-emitting devices that emit their respective narrow band light beams may be used for an illumination apparatus and image acquiring timings may be controlled to prevent color mixing or whole wavelength information (reflected light) obtained from an object may be simultaneously detected.

Furthermore, according to the methods for endoscopic treatment according to the aforementioned two embodiments, the light source apparatus 4 uses a xenon lamp, but a light-emitting diode (LED) or laser diode (LD) may also be used to emit white light or band-limited light.

Furthermore, according to the methods for endoscopic treatments according to the aforementioned two embodiments, in the narrow band light observation mode, narrow band light having a predetermined peak wavelength is radiated onto a subject as band-limited light, but light including narrow band light having a predetermined peak wavelength and having a broad range or light including not only narrow band light having a predetermined peak wavelength but also wideband light in other wavelength bands may also be radiated onto the subject as band-limited light.

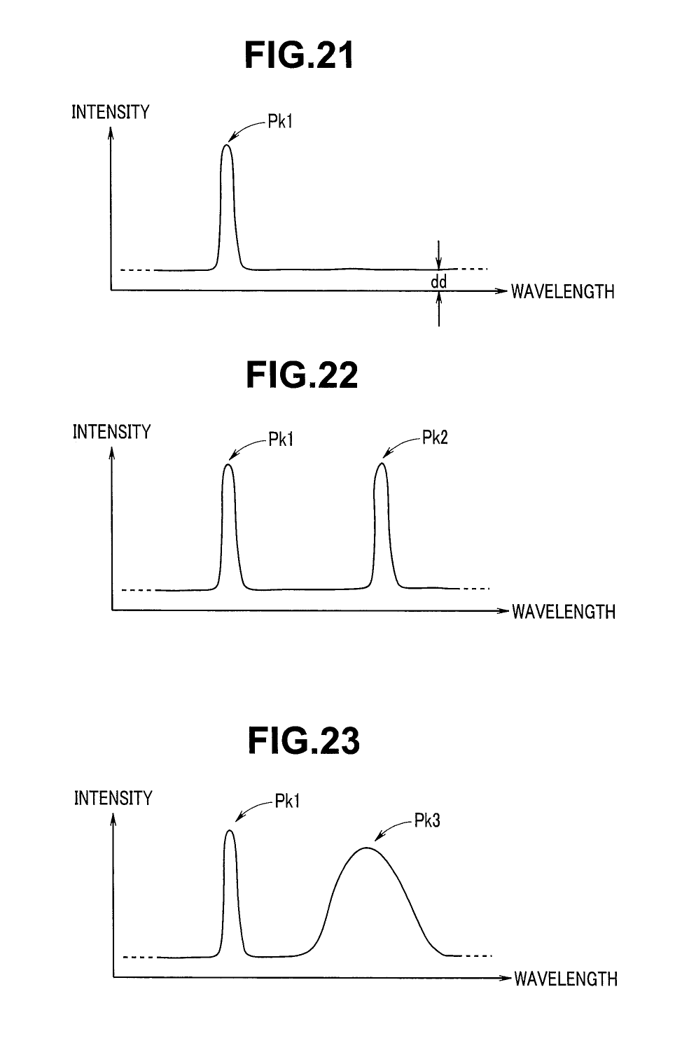

FIG. 21 to FIG. 23 are diagrams illustrating band-limited light. FIG. 21 is a diagram illustrating a relationship between wavelength and intensity of band-limited light including narrow band light having one predetermined peak wavelength and having a broad range. The band-limited light in FIG. 21 has a wavelength band including a peak Pk1, and has non-zero intensity dd in other wavelength bands.

FIG. 22 is a diagram illustrating a relationship between wavelength and intensity of band-limited light including narrow band light having two predetermined peak wavelengths and having a broad range. The band-limited light in FIG. 22 has a wavelength band including a peak Pk1 and a wavelength band including a peak Pk2 generated by a filter, and has non-zero intensity in other wavelength bands.

FIG. 23 is a diagram illustrating a relationship between wavelength and intensity of band-limited light including narrow band light having one predetermined peak wavelength and one wide band light. The band-limited light in FIG. 23 has a wavelength band including a peak Pk1 and wide band light including a peak Pk3, and has non-zero intensity in other wavelength bands. The light shown in FIG. 23 can be obtained by combining wide band light generated by fluorescent excitation light and narrow band light generated by a light-emitting diode (LED) or a laser diode (LD).

That is, not only narrow band light having a simple peak wavelength as described in the first and second embodiments but also the light described in FIG. 21 to FIG. 23 may be used as band-limited light for the methods for endoscopic treatment according to the aforementioned first and second embodiments.

The present invention is not limited to the aforementioned embodiments, but various modifications or changes or the like can be made without departing from the spirit and scope of the present invention.

What is claimed is:

1. A method for endoscopic treatment that performs treatment on a subject under an endoscope, the method comprising:
irradiating the subject with white light;
performing predetermined treatment on a living tissue of the subject after irradiation with the white light; and
switching from irradiation with the white light to irradiation of the subject with band-limited light having a predetermined peak wavelength according to a condition of bleeding from the living tissue in the predetermined treatment.

2. The method for endoscopic treatment according to claim 1, further comprising performing hemostasis treatment on a bleeding blood vessel of the living tissue using an electric knife or hemostasis forceps after radiation of the band-limited light.

3. The method for endoscopic treatment according to claim 2, further comprising switching from radiation of the band-limited light to radiation of the white light after the hemostasis treatment.

4. The method for endoscopic treatment according to claim 1, wherein the predetermined treatment is performed while radiating the band-limited light.

5. The method for endoscopic treatment according to claim 4, further comprising switching to radiation of the white light after performing the predetermined treatment while radiating the band-limited light.

6. The method for endoscopic treatment according to claim 1, further comprising performing preventive hemostasis treatment on the living tissue while radiating the band-limited light after the predetermined treatment.

7. The method for endoscopic treatment according to claim 1, wherein the predetermined treatment relates to endoscopic submucosal dissection, endoscopic mucosal resection, endoscopic sphincterotomy, treatment of an organ or treatment of brain surgery.

8. The method for endoscopic treatment according to claim 1, wherein the band-limited light has a peak wavelength in spectral characteristics in a red band of a visible range between a wavelength band including a maximum value and a wavelength band including a minimum value in hemoglobin light absorption characteristics of the living tissue of the subject.

9. The method for endoscopic treatment according to claim 8, wherein in radiation of the band-limited light, light including narrow band light of 585 nm to 630 nm is radiated.

10. A method for endoscopic treatment that performs treatment on a subject under an endoscope, the method comprising:
irradiating the subject with white light; and
switching, based on presence or absence of pulsatile bleeding after irradiation with the white light, from irradiation with the white light to irradiation of the subject with band-limited light having a peak wavelength in spectral characteristics in a red band of a visible range between a wavelength band including a maximum value and a wavelength band including a minimum value in hemoglobin light absorption characteristics of a living tissue of the subject.

11. A method for endoscopic treatment that performs treatment on a subject under an endoscope, the method comprising:
irradiating the subject with white light;
performing predetermined treatment on a living tissue of the subject after irradiation with the white light; and
switching, after the predetermined treatment, from irradiation with the white light to irradiation of the subject with band-limited light having a predetermined peak wavelength.

12. The method for endoscopic treatment according to claim 11, further comprising applying coagulation treatment or clip hemostasis to a non-bleeding blood vessel of the living tissue after radiation of the band-limited light.

13. The method for endoscopic treatment according to claim 12, further comprising switching from radiation of the band-limited light to radiation of the white light after the coagulation treatment or clip hemostasis.

14. The method for endoscopic treatment according to claim 11, wherein in the predetermined treatment, clipping treatment is performed on a hepatic artery of the subject to block a blood flow thereof.

15. The method for endoscopic treatment according to claim 14, further comprising performing resection treatment on the liver after radiation of the band-limited light.

16. The method for endoscopic treatment according to claim 11, wherein in the predetermined treatment, clipping treatment is performed on a cerebral aneurysm of the subject to block a blood flow thereof.

17. The method for endoscopic treatment according to claim 16, further comprising performing clipping treatment on the cerebral aneurysm again after radiation of the band-limited light.

18. The method for endoscopic treatment according to claim 11, wherein the predetermined treatment is biliary excretion treatment or urinary excretion treatment.

19. The method for endoscopic treatment according to claim 11, wherein the band-limited light has a peak wavelength in spectral characteristics in a red band of a visible range between a wavelength band including a maximum value and a wavelength band including a minimum value in hemoglobin light absorption characteristics of the living tissue of the subject.

20. The method for endoscopic treatment according to claim 19, wherein in radiation of the band-limited light, light including narrow band light of 585 nm to 630 nm is radiated.

21. A method for endoscopic treatment that performs treatment on a subject under an endoscope, the method comprising:
irradiating the subject with band-limited light;
performing predetermined treatment on a living tissue of the subject after irradiation with the white light; and
switching from irradiation with the band-limited light to irradiation of the subject with white light according to a condition of bleeding from the living tissue in the predetermined treatment.
